(19) United States
(12) Patent Application Publication
(10) Pub. No.: US 2024/0024427 A1
(43) Pub. Date: Jan. 25, 2024

(54) METHOD OF REDUCING INTRAOCULAR PRESSURE

(71) Applicant: INVEX THERAPEUTICS LTD, Perth (AU)

(72) Inventor: Alexandra SINCLAIR, Western Australia (AU)

(21) Appl. No.: 18/245,818

(22) PCT Filed: Sep. 17, 2021
(86) PCT No.: PCT/IB2021/058488

§ 371 Date: Mar. 17, 2023

(30) Foreign Application Priority Data
Sep. 18, 2020 (GB) 2014740.1

(51) Int. Cl. A61K 38/26; A61P 27/06

(52) U.S. Cl. CPC A61K 38/26; A61P 27/06

(57) ABSTRACT

The present invention provides a method of reducing intraocular pressure. In particular, the present invention provides a method of reducing intraocular pressure in a subject to treat a disorder associated with intraocular pressure, the method comprising administering a GLP-1 receptor agonist or a pharmaceutically acceptable salt thereof to the subject.

The present invention provides a method of reducing intraocular pressure. In particular, the present invention provides a method of reducing intraocular pressure in a subject to treat a disorder associated with intraocular pressure comprising administering a GLP-1 receptor agonist or a pharmaceutically acceptable salt thereof to the subject.

### BACKGROUND

Glaucoma is a progressive disease caused by raised intraocular pressure (IOP) that leads to damage to the optic nerve and ultimately irreversible blindness. Glaucoma is thought to affect over 70 million people worldwide, resulting in bilateral blindness in around 10% [1]. Intraocular pressure is determined by the balance between aqueous humour production inside the eye and aqueous humour drainage from the eye. IOP is typically 10-21 mmHg in healthy humans but it can drop as low as 0 mmHg in hypotony and can exceed 70 mmHg in some disease states. The rate at which raised intraocular pressure causes optic nerve damage depends on many factors, including the pressure and whether the glaucomatous damage is early or advanced. In general, pressures of 40-50 mmHg can cause rapid visual loss and pressures below this range usually cause damage over several years [2].

Reduction of intraocular pressure is the only proven method to try and help treat glaucoma. Such treatments can either act to reduce production of the fluid in the eye (aqueous humour from the ciliary body) or increase drainage from the eye. However, these approaches are symptomatic and not curative. Current approaches to reduce aqueous humour production are typically topical and utilise drugs such as Beta blockers (e.g. timolol, betaxolol), carbonic anhydrase inhibitors (e.g. dorzolamide) or alpha agonists (e.g. brimonidine), but these drugs are known to cause adverse ocular reactions and/or systemic side effects [3].

From the forgoing, it is evident that a need exists for new therapies for reducing intraocular pressure and treating associated conditions.

The incretin glucagon-like peptide-1 (GLP1) is a gut peptide predominantly secreted by the small intestine in response to food intake [4]. GLP-1 stimulates glucose-dependent insulin secretion and inhibits glucagon release, lowering blood glucose [5]. In addition, GLP-1 is synthesized in neurons of the nucleus tractus solitarius, which project to the hypothalamus and promote satiety and weight loss [6-9]. GLP-1 signals through the GLP-1 receptor, a class B G protein (heterotrimeric GTP-binding protein)-coupled receptor expressed in selected cell types within the central nervous system including the hypothalamus, hippocampus, olfactory cortex, circumventricular organs, hindbrain, and choroid plexus [10-12].

Recently, incretins and incretin receptor agonists have been shown to offer potential for reducing elevated intracranial pressure (ICP). In particular, it has been demonstrated that the GLP-1 receptor agonist, exendin-4, when dosed subcutaneously as an immediate release formulation in a conscious rat model reduced ICP when compared to a saline control [13-14].

Surprisingly, the Applicant has now found that GLP-1 receptor agonists are an effective therapy for reducing intraocular pressure and treating associated conditions such as glaucoma.

### SUMMARY OF INVENTION

In a first aspect, the present invention provides a GLP-1 receptor agonist or a pharmaceutically acceptable salt thereof for use in a method of reducing intraocular pressure (IOP) in a subject, wherein the method comprises administering the GLP-1 receptor agonist or a pharmaceutically acceptable salt thereof to the subject. Conveniently, the method of reducing IOP is used to treat a disorder associated with intraocular pressure, such as a disorder associated with elevated IOP.

In a further aspect of the present invention, the disorder associated with intraocular pressure can be Open-angle glaucoma, Ocular hypertension, Chronic open angle glaucoma, Primary angle closure glaucoma, Acute angle closure glaucoma, Angle closure glaucoma, Normal tension glaucoma (or Low tension glaucoma), Exfoliation syndrome, Pseudoexfoliation syndrome, Exfoliation glaucoma, Pigment dispersion syndrome, Primary congenital glaucoma, Nail-Patella syndrome, Secondary congenital glaucoma (such as Axenfeld-Rieger's Anomaly, Aniridia, Sturge-Weber syndrome), Childhood glaucoma secondary to intraocular surgery (such as removal of lens), Glaucoma secondary to retinopathy of prematurity, Pupil block glaucoma, Glaucoma associated with anterior segment dysgenesis, Glaucoma associated with disorders of the corneal endothelium, Iridocorneal endothelial syndrome (ICE syndrome), Chandler syndrome, Essential/Progressive Iris Atrophy, Iris Nevus/Cogan-Reese Syndrome, Glaucoma associated with disorders of the iris, Plateau iris, Glaucoma associated with disorders of the ciliary body, Glaucoma associated with disorders of the lens (such as phacomorphic glaucoma), Glaucoma associated with disorders of the retina (such as retinal detachment or Vogt-Koyanagi-Harada syndrome), Glaucoma associated with disorders of the vitreous, Glaucoma associated with disorders of the choroid, Glaucoma associated with elevated episcleral venous pressure (such as Sturge-Weber syndrome), Glaucoma associated with intraocular tumours, Glaucoma associated with ocular inflammation (such as HLA-B27-related acute anterior uveitis; Juvenile idiopathic arthritis-associated uveitis; Herpes virus-associated uveitis), Glaucoma associated with systemic inflammation (such as sarcoidosis associated uveitis), Glaucoma associated with systemic disease (such as sickle cell disease (SCD) and sickle cell trait (SCT) and neurofibromatosis), Steroid-induced glaucoma, Glaucoma associated with medications (such as topiramate), Glaucoma associated with intraocular haemorrhage, Neovascular glaucoma, Glaucoma associated with ocular trauma, Glaucoma secondary to angle recession, Glaucoma secondary to cyclodialysis cleft, Glaucoma after ocular surgery (such as vitroretinal surgery, corneal surgery, lens surgery and intravitreal injections of medication for age related macular degeneration) or Aqueous misdirection.

In another aspect of the present invention, the GLP-1 receptor agonist or a pharmaceutically acceptable salt thereof can reduce IOP in the subject by at least 5% from baseline, conveniently by at least 10% from baseline and more conveniently by at least 15%, by at least 20%, by at least 25%, or by at least 30% from baseline. The GLP-1 receptor agonist or a pharmaceutically acceptable salt thereof can also reduce IOP in a subject by at least 5%, conveniently by at least 10% and more conveniently by at least 15%, by at least 20%, by at least 25%, or by at least 30% from baseline for at least 3 hours after administration. The GLP-1 receptor agonist or a pharmaceutically acceptable salt thereof can also reduce IOP by at least 5% from baseline after 60 minutes from dosing, conveniently after 45 minutes from dosing, more conveniently after 30 minutes from dosing and even more conveniently after 10 minutes from dosing. The GLP-1 receptor agonist or a pharmaceutically acceptable salt thereof can also reduce IOP in the subject by at least 1 mmHg, conveniently at least 2 mmHg and more conveniently at least 3 mmHg from baseline.

In yet a further aspect of the present invention, the GLP-1 receptor agonist or a pharmaceutically acceptable salt thereof is administered by means of oral, sublingual, buccal, nasal, intra-arterial, intra-articular, intracardiac, intradermal, intramuscular, intraocular, intrathecal, intravenous, intravitreal, intraventricular, subcutaneous, subconjunctival, retrobulbar, peribulbar, intracameral or topical administration. Conveniently, the GLP-1 receptor agonist or a pharmaceutically acceptable salt thereof is administered topically to eye, subcutaneously or intravenously.

In a further aspect, the GLP-1 receptor agonist or a pharmaceutically acceptable salt thereof is administered at a dose of 1-50 μg, even more conveniently 5-40 μg, more conveniently 10-30 μg and even more conveniently 10-20 μg.

In a yet a further aspect, the GLP-1 receptor agonist or a pharmaceutically acceptable salt thereof can be administered once daily or twice daily.

In a further aspect, the GLP-1 receptor agonist or a pharmaceutically acceptable salt thereof is administered in combination with one or more therapeutic agents.

In a further aspect, the GLP-1 receptor agonists or a pharmaceutically acceptable salt thereof further provides a neuroprotective effect.

In yet a further aspect, the present invention provides a pharmaceutical composition comprising a GLP-1 receptor agonist or a pharmaceutically acceptable salt thereof and at least one pharmaceutically acceptable excipient, for use in a method of reducing intraocular pressure (IOP) in a subject to treat a disorder associated with intraocular pressure; wherein the method comprises administering the pharmaceutical composition to the subject.

### BRIEF DESCRIPTION OF THE FIGURES

FIG. 1 shows the mean change in intraocular pressure (in mmHg) from baseline (with SEM shown) to 12 weeks after treatment with exenatide or placebo—data includes both right and left eyes.

FIG. 2 shows the mean change in intraocular pressure (in mmHg) from baseline (with SEM shown) at 2.5, 6, 11 and 23 hours after treatment with exenatide or placebo—data includes both right and left eyes.

### DETAILED DESCRIPTION OF THE INVENTION

### Definitions

The disclosed methods may be understood more readily by reference to the following description which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Reference to a particular numerical value includes at least that particular value, unless the context clearly indicates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed methods which are for clarity, described herein in the context of separate embodiments may also be provided in combination in a single embodiment. Conversely, various features of the disclosed methods that are, for brevity, described in the context of a single embodiment, may also be provided separately or in any sub-combination.

As used herein, the singular forms “a”, “an”, and “the” include the plural.

The following abbreviations are used herein: glucagon-like peptide-1 (GLP-1), intraocular pressure (IOP), guansosine-5′-triphosphate (GTP), intracranial pressure (ICP), Iridocorneal endothelial syndrome (ICE syndrome), human leukocyte antigen B27 (HLA-B27), sickle cell disease (SCD), sickle cell trait (SCT), retinal ganglion cells (RGCs), standard deviation (SD), primary open-angle glaucoma (POAG), primary angle-closure glaucoma (PACG), glucagon-like peptide-1 receptor (GLP-1R), polyethylene glycol (PEG), European Summary of Product Characteristics (SmPC), U.S. Food and Drug Administration (FDA), optical coherence tomography (OCT), Detection of Apoptosing Retinal Cells (DARC), ethylenediaminetetraacetic acid (EDTA), butylated hydroxyanisole (BHA), idiopathic intracranial hypertension (IIH), magnetic resonance imaging (MRI), computerised tomography (CT), central corneal thickness (CCT) and body mass index (BMI).

As used herein, the term “treating” and like terms refer to reducing the severity and/or frequency of symptoms, eliminating symptoms and/or the underlying cause of said symptoms, reducing the frequency or likelihood of symptoms and/or their underlying cause, delaying, and/or slowing the progression of diseases and/or disorders, and improving or remediating damage cause, directly or indirectly, by the disease and/or disorders. In another embodiment, the term “treating” and like terms refer to preventing the progression of diseases and/or disorders.

As used herein, the phrase “therapeutically effective dose” refers to an amount of a composition comprising at least one active pharmaceutical ingredient, as described herein, effective to achieve a particular biological or therapeutic result such as, but not limited to, biological or therapeutic results disclosed, described or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of the benign or malignant disease or prevention of the development of the benign or malignant disease, as determined by any means suitable in the art.

As used herein, the phrasing “pharmaceutically acceptable salt” refers to those salts or compounds (for example GLP-1 receptor agonist described herein) which are, within the scope of sound medical judgement, suitable for use in contact with the tissues of human beings and animals without excessive toxicity, irritation, allergic response, or other problem or complication, commensurate with a reasonable benefit/risk ratio.

As used herein the term “intraocular pressure” refers to the fluid pressure of the eye.

As used herein the term “normal intraocular pressure” refers to two standard deviations above and below the population mean IOP. Conveniently, the mean IOP in normal adult populations is estimated at 15-16 mmHg, with a standard deviation of nearly 3.0 mmHg. More conveniently, the term “normal intraocular pressure” refers to IOP in a range of about 10-21 mmHg.

When values are expressed as approximations, by use of the antecedent “about”, it will be understood that the particular value forms another embodiment. As used herein and unless stated otherwise, it is to be understood that the term “about” is used synonymously with the term “approximately”. Illustratively and unless state otherwise, the use of the term “about” indicates values slightly outside the cited criteria values, namely ±10% (conveniently ±5%). Such values are thus encompassed by the scope of the claims reciting the terms “about” or “approximately”.

As used herein the term “elevated intraocular pressure” refers to IOP greater than two standard deviations above the population mean IOP. In other words an IOP greater than the upper limit of the “normal intraocular pressure”. Conveniently, the term “elevated intraocular pressure” refers to IOP greater than about 21 mmHg. In some embodiments, the IOP may be greater than about 25 mmHg, conveniently greater than about 30 mmHg and more conveniently greater than 35 mmHg.

As used herein the term “baseline” refers to the IOP measured prior to any treatment and is established by taking the mean of two IOP measurements (or three measurements if the first two measurements differ by more than 2 mmHg).

As used herein the term “neuroprotective effects” means non-IOP related prevention or delay of loss of retinal ganglion cells (RGCs) and their axons.

As used herein the phrase “pharmaceutically acceptable excipient” refers to a carrier medium which does not interfere with the effectiveness of the biological (or pharmaceutical) activity of the active ingredient and which is preferably not excessively toxic on the host at a concentration at which it is administered.

### Intraocular Pressure (OOP)

Intraocular pressure (IOP) refers to the fluid pressure of the eye. The IOP can be theoretically determined by the Goldmann equation, which is IOP=(F/C)+P, where F represents aqueous flow rate, C represents aqueous outflow, and P is the episcleral venous pressure. A change or fluctuation in any of these variables will inevitably alter the IOP. IOP is carefully regulated, and disturbances are often implicated in the development of pathologies such as glaucoma, uveitis, and retinal detachment. IOP exists as a fine-tuned equilibrium between the production and drainage of aqueous humour. Sudden increases in IOP can cause mechanical stress and ischemic effects on the retinal nerve fibre layer and on the blood flow from the optic nerve head to the retinal circulation, while sudden decrease in IOP can cause micro-bubbles to form from dissolved gases in microvasculature with resultant gas emboli and ischemic tissue damage. Chronic elevation of IOP has been implicated in the pathogenesis of glaucoma and other vision-damaging problems [15].

The intraocular pressure (IOP) in the population is approximately normally distributed with a right skew. The mean IOP in normal adult populations is estimated at 15-16 mmHg, with a standard deviation of nearly 3.0 mmHg. Traditionally, normal IOP has been defined as two standard deviations above and below the population mean IOP or about 10-21 mmHg, and any IOP above the upper limit of this range (i.e. greater than about 21 mmHg) is considered to be elevated. The level of IOP is a major risk factor for the development of glaucoma and its progression. For example, the risk of having glaucoma for with IOP measurements of 26 mmHg or greater is estimated to be 12 times higher than that for those with IOP within the normal range. IOP variation over a 24-hour cycle can be substantial and can be larger in glaucoma patients than in healthy individuals [16-17]. In particular, it has been shown that nocturnal supine IOP may be higher than diurnal sitting IOP [18].

Intraocular pressure is traditionally measured by Goldmann applanation tonometry, which gives an estimate of the pressure inside the eye based on the resistance to flattening of a small area of the cornea. Although Goldmann applanation tonometry is the reference standard for measuring IOP, intraobserver variability can occur. Some reports have suggested both the average intraobserver and interobserver variation in IOP can be around 1.6 mmHg. This intraobserver variation may be limited by measuring IOP twice, and a third time if the first two measurements differ by more than 2 mmHg [19]. Generally, the regulatory agencies require a 1.5 mmHg difference in IOP between two medications to declare a clinically significant difference. The standard deviation (SD) often used for pre-study unpaired sample size calculations is 3.5 mmHg [20].

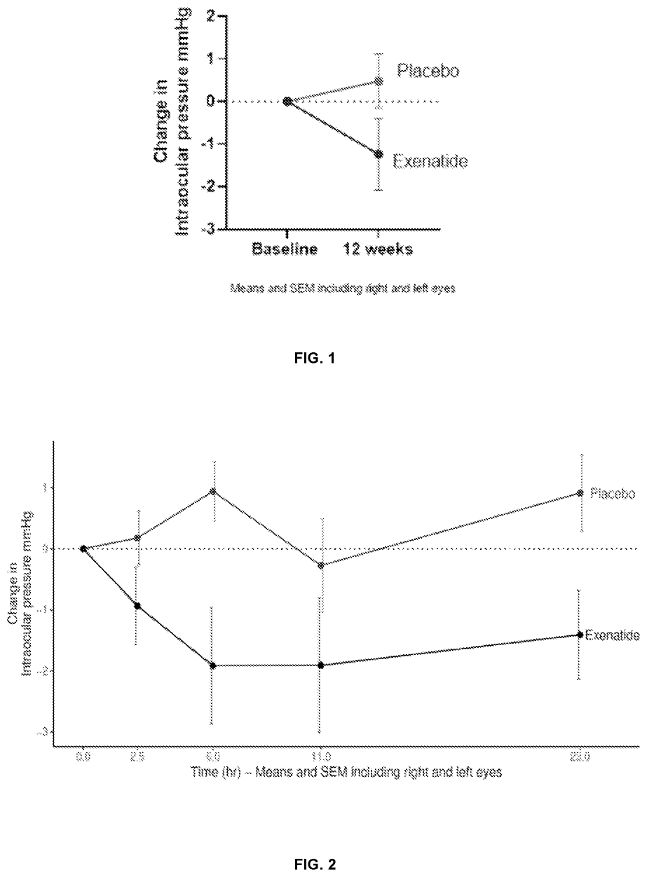

The Applicant has evaluated the response of intraocular pressure (IOP) to the administration of a GLP-1 receptor agonist as described herein as part of a sub-study of a pressure physiology study. The Applicant has surprisingly found that GLP-1 receptor agonists as described herein reduce intraocular pressure (IOP) in a subject. Further details of this study are described in the Examples and the results are shown in Tables 2-3 and FIGS. 1-2. These data suggest GLP-1 receptor agonists as described herein would be effective and beneficial in reducing intraocular pressure to treat diseases associated with intraocular pressure.

Without wishing to be bound to a theory, a GLP-1 receptor agonist or a pharmaceutically acceptable salt thereof as described herein is believed to reduce intraocular pressure by acting on the GLP-1 receptors at the ciliary body, thereby resulting in a reduction in sodium ion transport from the ciliary body and a reduction in the production of aqueous humour.

### Medical Use

In an aspect of the present invention, there is provided a GLP-1 receptor agonist or a pharmaceutically acceptable salt thereof as described herein for use in a method of reducing intraocular pressure (IOP) in a subject. In a further aspect of the present invention, there is provided a GLP-1 receptor agonist or a pharmaceutically acceptable salt thereof as described herein for use in a method of reducing intraocular pressure (IOP) in a subject to treat a disorder associated with intraocular pressure, wherein the method comprises administering the GLP-1 receptor agonist or a pharmaceutically acceptable salt thereof as described herein to the subject. In an embodiment, there is provided a GLP-1 receptor agonist, or a pharmaceutically acceptable salt thereof, as described herein for use in the treatment of a disorder associated with intraocular pressure. In an embodiment, there is provided a GLP-1 receptor agonist, or a pharmaceutically acceptable salt thereof, as described herein for use in the reduction of intraocular pressure in a subject. In an embodiment, there is provided a GLP-1 receptor agonist, or a pharmaceutically acceptable salt thereof, as described herein for use in the reduction of intraocular pressure in a subject suffering from a disorder associated with intraocular pressure. In an embodiment, there is provided a GLP-1 receptor agonist, or a pharmaceutically acceptable salt thereof, as described herein for use in the treatment of a disorder associated with elevated intraocular pressure. In an embodiment, there is provided a GLP-1 receptor agonist, or a pharmaceutically acceptable salt thereof, as described herein for use in the reduction of intraocular pressure in a subject suffering from a disorder associated with elevated intraocular pressure.

In a further aspect of the present invention, there is provided a method of reducing intraocular pressure (IOP) in a subject, the method comprising administering to the subject a GLP-1 receptor agonist or a pharmaceutically acceptable salt thereof as described herein. In a further aspect of the present invention, there is provided a method of reducing intraocular pressure (IOP) in a subject suffering from a disorder associated with intraocular pressure (IOP), the method comprising administering to the subject a GLP-1 receptor agonist or a pharmaceutically acceptable salt thereof as described herein. In an embodiment, there is provided a method of reducing intraocular pressure (IOP) in a subject suffering from a disorder associated with elevated IOP, the method comprising administering to the subject a GLP-1 receptor agonist, or a pharmaceutically acceptable salt thereof, as described herein.

In a further aspect of the present invention, there is provided use of a GLP-1 receptor agonist or a pharmaceutically acceptable salt thereof as described herein for the manufacture of a medicament for reducing intraocular pressure (IOP) in a subject. In a further aspect of the present invention, there is provided use of a GLP-1 receptor agonist or a pharmaceutically acceptable salt thereof as described herein for the manufacture of a medicament for reducing intraocular pressure (IOP) in a subject to treat a disorder associated with intraocular pressure (IOP). In an embodiment, there is provided the use of a GLP-1 receptor agonist, or a pharmaceutically acceptable salt thereof, as described herein for the manufacture of a medicament for reducing intraocular pressure (IOP) in a subject to treat a disorder associated with elevated intraocular pressure (IOP).

In one embodiment, the GLP-1 receptor agonist as described herein reduces elevated intraocular pressure (IOP). In one embodiment, the GLP-1 receptor agonist as described herein reduces IOP in a subject having a baseline IOP of greater than about 21 mmHg, or greater than about 25 mmHg, or greater than about 30 mmHg or greater than 35 mmHg.

In another embodiment, the GLP-1 receptor agonist as described herein reduces normal intraocular pressure (IOP). In one embodiment, the GLP-1 receptor agonist as described reduces IOP in a subject having a baseline IOP of about 10-21 mmHg.

As used herein the phrase “disorder associated with intraocular pressure (IOP)” is used to refer to a disorder characterized inter alia by physiologically damaging IOP and includes any disorder wherein a reduction in IOP would have a beneficial effect.

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing and/or slowing the progression of diseases and/or disorders, and improving or remediating damage cause, directly or indirectly, by the disease and/or disorders.

In a particular aspect of the present invention, the disorder associated with intraocular pressure is selected from Open-angle glaucoma, Ocular hypertension, Chronic open angle glaucoma, Primary angle closure glaucoma, Acute angle closure glaucoma, Angle closure glaucoma, Normal tension glaucoma (or Low tension glaucoma), Exfoliation syndrome, Pseudoexfoliation syndrome, Exfoliation glaucoma, Pigment dispersion syndrome, Primary congenital glaucoma, Nail-Patella syndrome, Secondary congenital glaucoma (such as Axenfeld-Rieger's Anomaly, Aniridia, Sturge-Weber syndrome), Childhood glaucoma secondary to intraocular surgery (such as removal of lens), Glaucoma secondary to retinopathy of prematurity, Pupil block glaucoma, Glaucoma associated with anterior segment dysgenesis, Glaucoma associated with disorders of the corneal endothelium, Iridocorneal endothelial syndrome (ICE syndrome), Chandler syndrome, Essential/Progressive Iris Atrophy, Iris Nevus/Cogan-Reese Syndrome, Glaucoma associated with disorders of the iris, Plateau iris, Glaucoma associated with disorders of the ciliary body, Glaucoma associated with disorders of the lens (such as phacomorphic glaucoma), Glaucoma associated with disorders of the retina (such as retinal detachment or Vogt-Koyanagi-Harada syndrome), Glaucoma associated with disorders of the vitreous, Glaucoma associated with disorders of the choroid, Glaucoma associated with elevated episcleral venous pressure (such as Sturge-Weber syndrome), Glaucoma associated with intraocular tumours, Glaucoma associated with ocular inflammation (such as HLA-B27-related acute anterior uveitis; Juvenile idiopathic arthritis-associated uveitis; Herpes virus-associated uveitis), Glaucoma associated with systemic inflammation (such as sarcoidosis associated uveitis), Glaucoma associated with systemic disease (such as sickle cell disease (SCD) and sickle cell trait (SCT) and neurofibromatosis), Steroid-induced glaucoma, Glaucoma associated with medications (such as topiramate), Glaucoma associated with intraocular haemorrhage, Neovascular glaucoma, Glaucoma associated with ocular trauma, Glaucoma secondary to angle recession, Glaucoma secondary to cyclodialysis cleft, Glaucoma after ocular surgery (such as vitroretinal surgery, corneal surgery, lens surgery and intravitreal injections of medication for age related macular degeneration) and Aqueous misdirection.

In a particular preferred embodiment, the disorder associated with intraocular pressure is selected from Ocular hypertension, Open-angle glaucoma, Primary angle closure glaucoma, Angle closure glaucoma and Normal tension glaucoma.

The GLP-1 receptor agonist, or a pharmaceutically acceptable salt thereof, as described herein can be used to prevent or manage a disorder associated with intraocular pressure (such as glaucoma), even when no damage to ocular tissue, vasculature or the optic nerve has occurred. In an embodiment, there is provided a GLP-1 receptor agonist, or a pharmaceutically acceptable salt thereof, as described herein for use in the reduction of intraocular pressure in a subject suffering from mild, early, or normal pressure glaucoma. Visual field loss may be quantified by parameters such as Humphrey mean deviation (see e.g. Yaqub, Community Eye Health (2012), 25(79-80), 1-8) wherein typically the mean deviation should be above −2 dB in healthy eyes; between 0 and −6 dB in early glaucoma; between −6 and −12 dB in moderate glaucoma; and below −12 dB in advanced or severe glaucoma (Mills et al, Am. J. Opthalmology (2006), 141(1), 24-30). In an embodiment, there is provided a GLP-1 receptor agonist, or a pharmaceutically acceptable salt thereof, as described herein for use in the reduction of intraocular pressure in a subject having a Humphrey Mean Deviation between +2 and −6 dB, such as between 0 and −6 dB.

Ocular hypertension is characterized by an IOP >21 mm Hg and lack of any glaucomatous changes of the optic disc or visual field defects. Therefore, in an embodiment, there is provided a GLP-1 receptor agonist, or a pharmaceutically acceptable salt thereof, as described herein for use in the reduction of intraocular pressure in a subject suffering from ocular hypertension. In an embodiment, there is provided Exenatide, or a pharmaceutically acceptable salt thereof, for use in the reduction of intraocular pressure in a subject suffering from ocular hypertension.

In an embodiment the GLP-1 receptor agonist or a pharmaceutically acceptable salt thereof as described herein reduces IOP in a subject by at least 5%, conveniently by at least 10% and more conveniently by at least 15% from baseline.

In another embodiment the GLP-1 receptor agonist or a pharmaceutically acceptable salt thereof as described herein reduces IOP in a subject by at least 5%, conveniently by at least 10%, and more conveniently by at least 15% from baseline for at least the first 3 hours after administration.

In another embodiment the GLP-1 receptor agonist or a pharmaceutically acceptable salt thereof as described herein reduces IOP in a subject by at least 5% for at least the first 4 hours after administration, conveniently for at least the first 8 hours after administration, more conveniently for at least the first 12 hours after administration, and even more conveniently for at least the first 24 hours after administration.

In another embodiment the GLP-1 receptor agonist or a pharmaceutically acceptable salt thereof (such as exenatide) as described herein reduces IOP in a subject by at least 5% 2.5 hours after administration, conveniently by at least 5% 6 hours after administration, 11 hours after administration, or 23 hours after administration.

In another embodiment the GLP-1 receptor agonist or a pharmaceutically acceptable salt thereof (such as exenatide) as described herein reduces IOP in a subject by at least 1 mm Hg 2.5 hours after administration, conveniently by at least 1 mm Hg 6 hours after administration, 11 hours after administration, or 23 hours after administration.

In another embodiment the GLP-1 receptor agonist or a pharmaceutically acceptable salt thereof (such as exenatide) as described herein reduces IOP in a subject by at least 10% 6 hours after administration, conveniently by at least 10% 11 hours after administration, or 23 hours after administration.

In another embodiment the GLP-1 receptor agonist or a pharmaceutically acceptable salt thereof (such as exenatide) as described herein reduces IOP in a subject by about 2 mm Hg 6 hours after administration, conveniently by about 2 mm Hg 11 hours after administration, or 23 hours after administration.

In an embodiment the GLP-1 receptor agonist or a pharmaceutically acceptable salt thereof as described herein reduces IOP in a subject by at least 5%, 10%, 15%, 20%, 25% or 30% from baseline after 60 minutes from dosing, conveniently after 45 minutes after dosing, more conveniently after 30 minutes from dosing and even more conveniently after 10 minutes from dosing. In an embodiment the GLP-1 receptor agonist or a pharmaceutically acceptable salt thereof as described herein when administered topically (e.g. via eye drops) reduces IOP in a subject by at least 5%, 10%, 15%, 20%, 25% or 30% from baseline after 60 minutes from dosing, conveniently after 45 minutes after dosing, more conveniently after 30 minutes from dosing and even more conveniently after 10 minutes from dosing. This may be particularly advantageous in treating a glaucoma characterized by a rapid rise in intraocular pressure, such as primary angle closure glaucoma and acute angle-closure glaucoma, where immediate treatment is needed to relieve symptoms and to prevent permanent loss of vision.

Therapy in glaucoma management aims to lower IOP to slow the rate of visual field deterioration. Target IOP is the upper limit of the IOP estimated to be compatible with a rate of progression sufficiently slow to maintain vision-related quality of life in the expected lifetime of the patient [16-17]. A clinical staging of glaucoma by optic nerve head evaluation and perimetric parameters (visual field parameters), allows a patient's eye to be categorized as having—mild, moderate or severe glaucomatous damage. An initial attempt should be made to achieve the following IOP range for both primary open-angle glaucoma (POAG) or primary angle-closure glaucoma (PACG) after iridotomy. In mild glaucoma the initial target IOP range could be kept as 15-17 mmHg, for moderate glaucoma 12-15 mmHg and in the severe stage of glaucomatous damage 10-12 mmHg [21].

It should be minded that these values are estimates only. The target IOP should be re-evaluated regularly and, additionally, when progression of disease is identified or when ocular or systemic comorbidities develop. Generally no single target IOP level is appropriate for every patient, so the target IOP is often estimated separately for each eye of every patient. Factors to consider when setting the target IOP include: the stage of glaucoma, IOP level before treatment (e.g. the lower the untreated IOP levels, the lower the target IOP should be), age and life expectancy, rate of progression during follow-up, the presence of other factors (e.g. exfoliation syndrome), the side effects and risk of treatment, and patient preference [16-17].

In an embodiment the GLP-1 receptor agonist or a pharmaceutically acceptable salt thereof as described herein reduces IOP in a subject by at least 1 mmHg, 2 mmHg, 3 mmHg, 4 mmHg, 5 mmHg, 6 mmHg, 7 mmHg, 8 mmHg, 9 mmHg or 10 mmHg from baseline.

In an embodiment the GLP-1 receptor agonist or a pharmaceutically acceptable salt thereof as described herein reduces IOP in a subject to less than 21 mmHg.

In an embodiment, the GLP-1 receptor agonist or a pharmaceutically acceptable salt thereof as described herein reduces IOP in a subject to less than 17 mmHg, or less than 15 mmHg, or less than 12 mmHg.

In another embodiment, the GLP-1 receptor agonist or a pharmaceutically acceptable salt thereof as described herein reduces IOP in a subject by at least 5%, 10%, 15%, 20%, 25% or 30% from baseline and to less than 21 mmHg.

In any of the above embodiments, there is also provided the equivalent embodiments wherein the method or treatment further comprises determining the baseline IOP of the subject prior to administration of the GLP-1 receptor agonist, and after a subsequent period of time measuring the change in IOP from baseline.

### GLP-1 Receptor Agonist

Glucagon-like peptide-1 (GLP-1) is an incretin hormone derived from the post-translational modification of proglucagon and secreted by gut endocrine cells. GLP-1 mediates its actions through a specific G protein-coupled receptor, namely GLP-1R. GLP-1 is characterized as a hormone that regulates glucose homeostasis.

As used herein, the term “GLP-1 receptor agonist”, also known as incretin mimetic, refers to compounds having glucagon-like peptide 1 (GLP-1) receptor agonist activity. It is to be understood that this includes the natural GLP-1 peptide (or either the forms it is primarily secreted as, namely GLP-1(7-37) and GLP-1(7-36)amide) and any analogues or derivatives thereof, which also have GLP-1 receptor agonist activity. The term “agonist”, as used herein, shall mean an agent (e.g. a ligand, or compound) that by virtue of binding to a GLP-1 receptor activates the receptor so as to elicit an intracellular response mediated by the receptor.

The term “analogue” includes a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide. In one embodiment an analogue exhibits at least 40% (conveniently at least 50%, 60%, 70%, 80% and 90%) homology to the amino acid sequence of the native peptide. In one embodiment an analogue comprises less than 10 modifications (substitutions, deletions, additions). In another embodiment an analogue comprises less than 6 modifications (substitutions, deletions, additions) relative to the native peptide. In another embodiment an analogue comprises less than 5 modifications (substitutions, deletions, additions) relative to the native peptide. In another embodiment an analogue comprises less than 4 modifications (substitutions, deletions, additions) relative to the native peptide. In another embodiment an analogue comprises less than 3 modifications (substitutions, deletions, additions) relative to the native peptide. In another embodiment an analogue comprises less than 2 modifications (substitutions, deletions, additions) relative to the native peptide. In another embodiment an analogue comprises only a single modification (substitutions, deletions, additions) relative to the native peptide.

The term “derivative” as used herein in relation to a parent peptide/protein or analogue means a chemically modified parent peptide/protein or analogue thereof, wherein at least one substituent is not present in the parent peptide/protein or an analogue thereof. For example, a derivative would include a parent peptide/protein or an analogue thereof which has been covalently modified. Typical modifications include amides, carbohydrates, alkyl groups, acyl groups, esters, PEGylations, immunoglobulins (such as IgG4), plasma proteins (such as α, β, γ globulins, glycoprotein, lipoprotein and albumin, conveniently albumin) and the like. Such entities can be directly conjugated to the parent peptide/protein or analogue thereof or be conjugated via a suitable linker.

Exemplary types of GLP-1 receptor agonists include exendins, exendin analogs, GLP-1(7-36)amide, GLP-1(7-36)amid analogs, GLP-1(7-37), GLP-1(7-37) analogs, and the like.

The term “exendin” includes naturally occurring (or synthetic versions of naturally occurring) exendin peptides that are found in the salivary secretions of some beaded lizards (Heloderma). Exendins of particular interest include exendin-3, exendin-4 and exenatide, especially exendin-4 and exenatide. Exendin-4 is a 39 amino acid peptide secreted by the salivary glands of the desert Gila monster (Heloderma suspectum). Exendin-4 is a potent agonist of mammalian GLP-1R and shares around 50% sequence homology with human GLP-1. Exendin-3 is a peptide found in the saliva of the Mexican beaded lizard (Heloderma horridum) and differs from exendin-4 by two amino acid substitutions, Gly2-Glu3 in place of Ser2-Asp3, but is otherwise identical. Exenatide is a synthetic version of exendin-4.

The term “exendin analog” refers to peptides or other compounds which elicit a biological activity of an exendin reference peptide, preferably having a potency equal to or better than the exendin reference peptide (e.g. exendin-4), or within five orders or magnitude (plus or minus) of potency compared to the exendin reference peptide, when evaluated by art-known measures such as receptor binding and/or competition studies (Hargrove D M, Kendall E S, Reynolds J M et al., Biological activity of AC3174, a peptide analog of exendin-4, Regulatory Pept. 2007; 141: 113-119). In one embodiment, the term “exendin analog” refers to a peptide having at least 75% sequence identity to exendin-4. Exendin analogs include lixisenatide (Sanofi-Aventis) and albenatide (ConjuChem, Inc.). Lixisenatide is a 44-amino acid peptide based on the structure of exendin-4, with modifications consisting of a deletion of a proline residue and addition of six lysine residues at the C-terminal. Albenatide is a 40-amino acid peptide based on the structure of exendin-4, with modifications consisting of an additional lysine residue conjugated to recombinant human albumin.

As mentioned above, GLP-1 is secreted primarily in two forms, GLP-1(7-37) and GLP-1(7-36)amide. The intracellular precursor to GLP-1, GLP-1(1-37), is cleaved from proglucagon, and the first six amino acids are subsequently removed from the N-terminus to form bioactive peptides. About 80% of truncated GLP-1 is amidated to form GLP-1(7-36)amide, the predominant secreted form of GLP-1, whereas the remainder is released as GLP-1(7-37). GLP-1(7-37) and GLP-1(7-36)amide have a very short physiological half-life of less than 2 minutes. This is due to the rapid cleavage of the amide bond of alanine in position 8 at the N-terminal by dipeptidyl peptidase-4 (DPP-IV), resulting in two truncated inactive forms, GLP-1(9-36) and GLP-1(9-37).

The term “GLP-1(7-36) analogs” refers to peptides or other compounds which elicit an enhanced DPP-IV enzymatic stability compared to native GLP-1(7-36)amide and/or which elicit a biological activity of GLP-1(7-36)amide, preferably a potency equal to or better than GLP-1(7-36)amide, or within five orders of magnitude (plus or minus) of potency compared to GLP-1(7-36)amide. GLP-1(7-36) analogs of particular interest include albiglutide (GlaxoSmithKline). Albiglutide consists of two modified GLP-1(7-36) fused in tandem to human serum albumin. The peptide sequence has a glycine to alanine replacement at position 8.

The term “GLP-1(7-37) analogs” refers to peptides or other compounds which elicit an enhanced DPP-IV enzymatic stability compared to native GLP-1(7-37) and/or which elicit a biological activity of GLP-1(7-37), preferably a potency equal to or better than GLP-1(7-37), or within five orders of magnitude (plus or minus) of potency compared to GLP-1(7-37)amide. GLP-1(7-37) analogs include liraglutide (Novo Nordisk), dulaglutide (Eli Lilly and Co.) and semaglutide (Novo Nordisk). Liraglutide is 97% homologous to native human GLP-1(7-37) and contains a substitution of arginine for lysine at position 34. Liraglutide is made by attaching a C-₁₆ fatty acid (palmitic acid) with a glutamic spacer on the remaining lysine residue at position 26 of the peptide precursor. Dulaglutide consists of a modified human GLP-1 analog sequence covalently attached to each of the two chains of a modified human immunoglobulin G4 (IgG4) heavy chain fragment (Fc) via a flexible 16 amino acid peptide with three Gly-Gly-Gly-Ser repeat sequences and an Ala residue. The GLP-1 analog is approximately 90% homologous to native GLP-1(7-37) with amino acid substitutions at position 8, 22 and 36 by a small peptide linker. The GLP-1 analog portion of dulaglutide is approximately 90% homologous to native human GLP-1(7-37) by substituting alanine for glycine at position 8, glycine for glutamic acid at position 22 and alanine for glycine at position 36. Semaglutide is chemically similar to liraglutide, with the inclusion of two structural modifications. The first is replacement of glycine with the non-proteinogenic amino acid 2-aminoisobutyric acid (Aib) at position 2. The second is the attachment of octadecanoic diacid to the side chain of lysine at position 26 through a short polyethylene glycol (PEG) spacer and a γ-glutamic acid linker.

It is understood that the biological half-life of GLP-1 receptor agonists or pharmaceutically acceptable salts thereof vary. For example, according to the relevant European Summary of Product Characteristics (SmPC) or FDA product label publications, the reported mean plasma terminal half-life for a range of marketed GLP-1 receptor agonists are as follows:

| | Mean plasma terminal | |
|---|---|---|
| Product | half-life | Source |
| Exenatide | 2.4 hours | Byetta product label, U.S. FDA, |
| | | Issued 28 Apr. 2005 |
| Lixisenatide | Approximately 3 hours | SmPC for Lyxumia (date of first |
| | | authorization 1 Feb. 2013) |
| Liraglutide | Approximately 13 hours | SmPC for Victoza (date of first |
| | | authorization 30 Jun. 2009) |
| Dulaglutide | 4.5 to 4.7 days | SmPC for Trulicity (date of first |
| | | authorization 21 Nov. 2014) |
| Semaglutide | Approximately 1 week | SmPC for Ozempic (date of first |
| | | authorization 8 Feb. 2018) |
| Albiglutide | Approximately 5 days | SmPC for Eperzan (date of first |
| | | authorization 21 Mar. 2014) |

In a preferred embodiment, the GLP-1 receptor agonist or the pharmaceutically acceptable salt thereof is selected from exendin-4, exenatide, liraglutide, lixisenatide, albiglutide, dulaglutide, semaglutide or a combination thereof.

In a particular embodiment, the GLP-1 receptor agonist is selected from exendin-4, exenatide and lixisenatide or a pharmaceutically acceptable salt thereof.

In a more preferred embodiment, the GLP-1 receptor agonist is exendin-4 or exenatide or a pharmaceutically acceptable salt thereof.

The GLP-1 receptor agonists or pharmaceutically acceptable salt thereof may be used in various solid forms, all of which are included within the scope of the invention. These include amorphous or crystalline forms, and anhydrous forms as well as solvates of hydrates.

The GLP-1 receptor agonist may be used as a pharmaceutically acceptable salt, such as a pharmaceutically acceptable acid addition salt formed through reaction with a suitable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid.

### Administration

The GLP-1 receptor agonist or a pharmaceutically acceptable salt as described herein may be administered by means of oral, sublingual, buccal, nasal, intra-arterial, intra-articular, intracardiac, intradermal, intramuscular, intraocular, intrathecal, intravenous, intravitreal, subcutaneous, subconjunctival, retrobulbar, peribulbar, intracameral or topical administration.

In an embodiment, the GLP-1 receptor agonist or a pharmaceutically acceptable salt as described herein is administered topically to the eye.

In another embodiment, the GLP-1 receptor agonist or a pharmaceutically acceptable salt thereof as described herein is administered by means of subcutaneous administration.

In a yet another embodiment, the GLP-1 receptor agonist or a pharmaceutically acceptable salt as described herein is administered by means of intravenous administration.

In an embodiment, the GLP-1 receptor agonist or a pharmaceutically acceptable salt as described herein is administered at a dose of 1-50 μg, conveniently 5-40 μg, more conveniently 10-30 μg and even more conveniently 10-20 μg.

In another embodiment, the GLP-1 receptor agonist or a pharmaceutically acceptable salt thereof as described herein is administered once daily.

In another embodiment, the GLP-1 receptor agonist or a pharmaceutically acceptable salt thereof as described herein is administered twice a day.

In an embodiment, the GLP-1 receptor agonist or a pharmaceutically acceptable salt thereof as described herein is administered within two hours prior to sleep onset.

In another embodiment, the GLP-1 receptor agonist or a pharmaceutically acceptable salt thereof as described herein is administered once daily within two hours prior to sleep onset.

In an embodiment, the GLP-1 receptor agonist, or a pharmaceutically acceptable salt thereof, as described herein is administered over a period of at least 1 week, such as at least 2, 3, 4, 5, 6, 7, 8, 9, 10, 11, or 12 weeks. In an embodiment, the GLP-1 receptor agonist, or a pharmaceutically acceptable salt thereof, as described herein is administered via once or twice daily dosing over a period of at least 1 week, such as at least 2, 3, 4, 5, 6, 7, 8, 9, 10, 11, or 12 weeks.

In an embodiment, the GLP-1 receptor agonist, or a pharmaceutically acceptable salt thereof, as described herein is administered over a period of at least 24, 36, 48, or 52 weeks. In an embodiment, the GLP-1 receptor agonist, or a pharmaceutically acceptable salt thereof, as described herein is administered via once or twice daily dosing over a period of at least 24, 36, 48, or 52 weeks.

In an embodiment, Exenatide, or a pharmaceutically acceptable salt thereof, is administered over a period of at least 1 week, such as at least 2, 3, 4, 5, 6, 7, 8, 9, 10, 11, or 12 weeks. In an embodiment, Exenatide, or a pharmaceutically acceptable salt thereof, is administered via once or twice daily dosing over a period of at least 1 week, such as at least 2, 3, 4, 5, 6, 7, 8, 9, 10, 11, or 12 weeks. In an embodiment, Exenatide, or a pharmaceutically acceptable salt thereof, is administered at a daily dose of about g to about 20 μg over a period of at least 1 week, such as at least 2, 3, 4, 5, 6, 7, 8, 9, 10, 11, or 12 weeks. In an embodiment, Exenatide, or a pharmaceutically acceptable salt thereof, is administered at a daily dose of 10 μg or 20 μg over a period of at least 1 week, such as at least 2, 3, 4, 5, 6, 7, 8, 9, 10, 11, or 12 weeks. In an embodiment, Exenatide, or a pharmaceutically acceptable salt thereof, is administered at a daily dose of 10 μg or 20 μg over a period of at least 24, 36, 48, or 52 weeks.

In an embodiment, Exenatide, or a pharmaceutically acceptable salt thereof, is administered over a period of at least 10, 11, or 12 weeks. In an embodiment, Exenatide, or a pharmaceutically acceptable salt thereof, is administered at a daily dose of 10 μg or 20 μg over a period of at least 10, 11, or 12 weeks. In an embodiment, Exenatide, or a pharmaceutically acceptable salt thereof, is administered twice weekly over a period of at least 10, 11, or 12 weeks. In an embodiment, Exenatide, or a pharmaceutically acceptable salt thereof, is administered once weekly over a period of at least 10, 11, or 12 weeks.

In an embodiment, Exenatide, or a pharmaceutically acceptable salt thereof, is administered topically (e.g. via eye drops) over a period of at least 10, 11, or 12 weeks. In an embodiment, Exenatide, or a pharmaceutically acceptable salt thereof, is administered topically (e.g. via eye drops) over a period of at least 24, 36, or 48 weeks. In an embodiment, Exenatide, or a pharmaceutically acceptable salt thereof, is administered topically (e.g. via eye drops) at a daily dose of 10 μg or 20 μg over a period of at least 10, 11, or 12 weeks.

As the treatment of disorders associated with intraocular pressure with a GLP-1 receptor agonist as described herein typically results in treatment of the symptoms via a reduction in IOP, rather than a curing of the disorder altogether, chronic dosing regimens may be required. Such chronic dosing regimens may include more than one phase of dosing—for example, an initial treatment phase in order to reduce the IOP to a desirable level and a subsequent maintenance phase to keep the IOP at this level and prevent recurrence of the disorder associated with intraocular pressure. Typically the treatment phase involves administering a higher dose of the GLP-1 receptor agonist and the maintenance phase involves administering a lower dose of the GLP-1 receptor agonist. In an embodiment, a GLP-1 receptor agonist (such as Exenatide), or a pharmaceutically acceptable salt thereof, is administered over a period of at least 10, 11, or 12 weeks, wherein the dosage regimen comprises a treatment phase and a maintenance phase.

Conveniently, the treatment phase comprises daily dosing of the GLP-1 receptor agonist (e.g. exenatide). Conveniently, the treatment phase comprises dosing of the GLP-1 receptor agonist (e.g. exenatide) at a dose of at least 10 μg, such as at least 20 μg. In an embodiment, the treatment phase lasts at least 5 weeks, such as at least 6 weeks. In an embodiment, the treatment phase lasts until the subject has an IOP of less than 21 mmHg, such as less than 20 mmHg, less than 19 mmHg, less than 18 mmHg, less than 17 mmHg, less than 15 mmHg, less than 12 mmHg, or less than 10 mmHg. In an embodiment, the treatment phase lasts until it reduces the IOP in the subject by at least 5%, 10%, 15%, 20%, 25% or 30% from baseline. In an embodiment, the treatment phase lasts until it reduces the IOP in the subject by at least 30% from baseline. In an embodiment, the treatment phase lasts until it reduces the IOP in the subject by at least 30% from baseline and to less than 21 mmHg.

Conveniently, the maintenance phase comprises once, twice, or thrice weekly dosing of the GLP-1 receptor agonist (e.g. exenatide). Conveniently, the maintenance phase comprises dosing of the GLP-1 receptor agonist (e.g. exenatide) at a dose of less than 20 μg, such as less than 10 μg, or less than 5 μg. In an embodiment, the maintenance phase lasts at least 5 weeks, such as at least 6 weeks, at least 10 weeks, or at least 12 weeks. In an embodiment, the maintenance phase maintains the IOP of the subject at less than 21 mmHg, such as less than 20 mmHg, less than 19 mmHg, less than 18 mmHg, less than 17 mmHg, or less than 15 mmHg.

In an embodiment, the GLP-1 receptor agonist or a pharmaceutically acceptable salt thereof as described herein, after subcutaneous administration to a subject in need of treatment thereof, produces a geometric mean (or geometric maximum) plasma concentration of the GLP-1 receptor agonist at 1 hour administration of at least 20 μg/ml.

In one embodiment, the GLP-1 receptor agonist or a pharmaceutically acceptable salt as described herein is administered to a human.

In another embodiment, the subject is an animal, conveniently a mammal and more conveniently a mammal and selected from a productive livestock (such as a sheep, cattle, horse, goat or pig), domestic animal or pet (such as a mouse, rat, hamster, guinea pig, dog or cat), and zoo animal (such as a primate, elephant, big cat, giraffe, antelope, zebra or lama).

### Neuroprotective Effect

Glaucoma is an optic neuropathy, specifically a neurodegenerative disease characterized by loss of retinal ganglion cells (RGCs) and their axons. Clinical evidence indicates that risk factors other than IOP may also intervene in the pathogenesis of the neuronal damage of glaucoma. Neuroprotectants are an alternative strategy for treatment of glaucoma. Neuroprotectants provide non-IOP-related intervention that can prevent or delay glaucomatous neurodegeneration, independently of IOP [22]. RGC apoptosis in the eye and therefore degree of neuroprotection may be monitored inter alia with perimetry (visual fields), electrophysiological and optical coherence tomography (OCT) measurements as well as the Detection of Apoptosing Retinal Cells (DARC) technique, which quantifies in vivo the death of ganglion cells. The methods and procedures for implementing these monitoring techniques are known to the person skilled in the art [22].

A number of therapies such as brimonidine and dorzolamide, claim to be dual acting, that is, provide both IOP lowering and neuroprotective effects. However, evidence for their dual efficacy is inconclusive. In particular, there is evidence that the neuroprotection conferred by these agents may be due to the IOP reduction rather than their direct neuroprotective properties [23]. Thus there remains a need for dual acting therapies that reduce IOP and, independent of IOP, provide a neuroprotective effect.

In an embodiment, the GLP-1 receptor agonists as described herein are dual acting, that is, they reduce IOP and, independently of IOP, further provide a neuroprotective effect. In an embodiment, the GLP-1 receptor agonists as described herein reduce IOP and prevent progressive loss of retinal ganglion cells (as determined by OCT or DARC methods).

### Pharmaceutical Composition

In one aspect, the present invention provides a pharmaceutical composition comprising a GLP-1 receptor agonist or a pharmaceutically acceptable salt thereof and at least one pharmaceutically acceptable excipient, for use in a method of reducing intraocular pressure (IOP) in a subject to treat a disorder associated with intraocular pressure; wherein the method comprises administering the pharmaceutical composition to the subject. In an embodiment, there is provided a pharmaceutical composition comprising a GLP-1 receptor agonist, or a pharmaceutically acceptable salt thereof, and at least one pharmaceutically acceptable excipient, for use in the treatment of a disorder associated with intraocular pressure. In an embodiment, there is provided a pharmaceutical composition comprising a GLP-1 receptor agonist, or a pharmaceutically acceptable salt thereof, and at least one pharmaceutically acceptable excipient, for use in the treatment of a disorder associated with elevated intraocular pressure. In an embodiment, there is provided a pharmaceutical composition comprising a GLP-1 receptor agonist, or a pharmaceutically acceptable salt thereof, and at least one pharmaceutically acceptable excipient, for use in the reduction of intraocular pressure in a subject. In an embodiment, there is provided a pharmaceutical composition comprising a GLP-1 receptor agonist, or a pharmaceutically acceptable salt thereof, and at least one pharmaceutically acceptable excipient, for use in the reduction of intraocular pressure in a subject suffering from a disorder associated with intraocular pressure.

The pharmaceutical composition as described herein may be administered by means of oral, sublingual, buccal, nasal, intra-arterial, intra-articular, intracardiac, intradermal, intramuscular, intraocular, intrathecal, intravenous, intravitreal, subcutaneous, subconjunctival, retrobulbar, peribulbar, intracameral or topical administration.

In one embodiment, the pharmaceutical composition comprising a GLP-1 receptor agonist as described herein is configured for subcutaneous administration. The pharmaceutical composition may be in the form of sterile injectable solution in one or more aqueous or non-aqueous non-toxic parenterally-acceptable buffer systems, diluents, solubilizing agents, co-solvents, or carriers.

In one embodiment, the pharmaceutical composition comprising a GLP-1 receptor agonist as described herein is formulated as an immediate release formulation.

In one embodiment, the pharmaceutical composition comprising at a GLP-1 receptor agonist or a pharmaceutically acceptable salt thereof and at least one pharmaceutically acceptable excipient is configured for topical administration to the eye.

The term “topical”, when used herein to characterize the delivery, administration or application of a GLP-1 receptor agonist or pharmaceutical composition as described herein, is meant to specify that the GLP-1 receptor agonist or pharmaceutical composition as described herein is delivered, administered or applied directly to the site of interest (i.e., to the eye) for a localized effect.

Examples of topical formulations include, but are not limited to, drops (such as eye drops), lotions, sprays, hydrogels, aerosols, foams, ointments, creams, gels, pastes and the like.

In another embodiment, the pharmaceutical composition further comprises additives such as diluents, buffering agents, dispersing or wetting agents, preservatives, anti-foaming agent, antioxidants, rheology modifying agent (such as viscosity modifier(s)), tonicity agents and combinations thereof.

In another embodiment, the pharmaceutical composition is preservative free. Conveniently, the pharmaceutical composition is a preservative free eye drop composition.

Inert diluents may be sucrose, sorbitol, sugar, mannitol, microcrystalline cellulose, starches, calcium carbonate, sodium chloride, lactose, calcium phosphate, calcium sulphate or sodium phosphate.

Buffering agents include citric acid, acetic acid, lactic acid, hydrogenophosphoric acid, diethylamine, sodium hydroxide and tromethane.

Dispersing or wetting agents include naturally occurring phosphatides (e.g. lecithin or soybean lecithin), condensation products of ethylene oxide with fatty acids or with long chain aliphatic alcohols (e.g. polyoxyethylene stearate, polyoxyethylene sorbitol monoleate, and polyoxyethylene sorbitan monooleate).

Preservatives may be added to a composition of the invention to prevent microbial contamination that can affect stability of the formulation and/or cause infection in the patient.

Anti-foaming agents usually facilitate manufacture of pharmaceutical compositions, they dissipate foam by destabilizing the air-liquid interface and allow liquid to drain away from air pockets. Examples of anti-foaming agents include simethicone, dimethicone, ethanol and ether.

Suitable antioxidants include, but are not limited to butylated hydroxyanisole (BHA), butylated hydroxytoluene, ascorbic acid, sodium metabisulphite, propyl gallate, sodium thiosulphate, alphatocopherol, ascorbic acid, retinoic acid, lutein, derivatives, precursors or prodrugs thereof, and mixtures of two or more thereof.

Tonicity agent may be selected from the group consisting of a salt, a sugar, a sugar alcohol, a glycol, a carbamide and mixtures of two or more thereof.

The size of the dose required for the therapeutic or prophylactic treatment of a particular disease state will necessarily be varied depend on the host treated, the route of administration and the severity of the illness being treated. Accordingly, the optimum dosage may be determined by the practitioner who is also treating any particular patient. Suitably, unit dosage forms will contain about 0.01-100 μg, conveniently 1-50 μg, more conveniently 5-40 μg, even more conveniently 10-30 μg and still more conveniently 10-20 μg of a GLP-1 receptor agonist as described herein.

### Combination Therapies

A GLP-1 receptor agonist or a pharmaceutically acceptable salt as described herein may be administered alone as a sole therapy or can be administered in addition with one or more other substances and/or treatments. Such conjoint treatment may be achieved by way of simultaneous sequential or separate administration of the individual components of the treatment.

For example, therapeutic effectiveness may be enhanced by administration of an adjuvant (i.e. by itself the adjuvant may only have minimal therapeutic benefit, but the combination with another therapeutic agent, the overall therapeutic benefit to the individual is enhanced). Or, by way of example only, the benefit experienced by the individual may be increased by administering the GLP-1 receptor agonist or a pharmaceutically acceptable salt thereof with another therapeutic agent (which also includes a therapeutic regimen) that also has therapeutic benefit. By way of example only, in glaucoma therapy, increase therapeutic benefit may result by also providing the individual with another therapeutic agent for glaucoma. Or, the additional therapy or therapies may include, but are not limited to, laser treatment (such as selective laser trabeculoplasty, cyclodiode laser treatment and laser iridotomy) and surgery (such as trabeculectomy, viscocanlostomy, deep sclerectomy and trabecular stent bypass).

In the instances where the GLP-1 receptor agonist or a pharmaceutically acceptable salt thereof is administered in combination with other therapeutics, the GLP-1 receptor agonist or a pharmaceutically acceptable salt thereof need not be administered via the same route as other therapeutic agents, and may, because of different physical and chemical characteristics be administered by a different route. For example, the GLP-1 receptor agonist or a pharmaceutically acceptable salt thereof may be subcutaneous to generate and maintain good blood levels thereof, while the other therapeutic agent may be administered topically. The initial administration may be made according to established protocols known in the art and/or described herein, and then, based upon observed effects, the dosage, modes of administration and times of administration can be modified by the skilled person.

The particular choice of other therapeutic will depend upon the diagnosis of the attending physicians and their judgement of the condition of the individual and the appropriate treatment protocol. In some embodiments, the additional agent is for the treatment of glaucoma.

In some embodiments, the GLP-1 receptor agonist is administered in combination with one or more therapeutic agents selected from adrenergic agonists (such as apraclonidine and brimonidine), adrenergic antagonists (such as betaxolol, carteolol, levobunolol, metipranolol, timolol maleate and timolol hemihydrate), carbonic anhydrase inhibitors (such as dorzolamide, brinzolamide, methazolamide and acetazolamide), cholinergic agents (such as acetylcholine, carbachol and pilocarpine), prostaglandin derivates (such as bimatoprost, latanoprost, tafluprost, latanoprostene bunod and travoprost), phenoxyacetic acid derivatives, steroid antagonists (such as mifepristone), atrial natriuretic peptides, angiotensin converting enzyme inhibitors (such as enalapril and captopril), ocular hypotensive lipids, neuroprotective agents (such as coenzyme Q10 and citicoline), Rho kinase inhibitors (such as netarsudil and ripasudil), nitric oxides and beta blockers. In one embodiment, the GLP-1 receptor agonist is administered in combination with one or more beta blockers.

### Kits

In one embodiment, the GLP-1 receptor agonist or a pharmaceutically acceptable salt thereof, pharmaceutical compositions and methods described herein provide kits for the treatment of disorders, such as the one described herein. These kits comprise a GLP-1 receptor agonist or a pharmaceutically acceptable salt thereof as described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the GLP-1 receptor agonist or a pharmaceutically acceptable salt as described herein, and/or which describe dosing, administration, side effects, drug interactions, or other information useful to the heath care provider. Such information may be based on the result of various studies, for example, studies using experimental animals involving in vivo models and studies based on clinical trials. Kits described herein can be provided, marketed and/or promoted to health providers, including physicians, nurses, pharmacists, formulary officials, and the like. Kits may also, in some embodiments, be marketed directly to the consumer.

### Examples

As part of a sub-study of the clinical trial titled “IIH Pressure: The acute and chronic effects of gut neuropeptides on intracranial pressure regulation” the Applicant evaluated the response of intraocular pressure (IOP) in participants to the administration of exenatide.

### Methods

### Subjects

Female Idiopathic Intracranial Hypertension (IIH) patients aged between 18 and 60 years, diagnosed according to the modified Dandy criteria with active disease (papilloedema [Frisen grade≥1], significantly raised ICP>25cmH₂O) and no evidence of venous sinus thrombosis (magnetic resonance imaging (MRI) or computerised tomography (CT) imaging and venography as noted at diagnosis), and not known to have diseases related to intraocular pressure.

### Visits

Patients attended a baseline visit fasted. At the baseline visit patients were randomised to either an Exenatide, or a control arm (saline placebo) group.

Following a baseline measurement of Intraocular pressure (IOP) in one eye (measured supine utilizing an iCare Pro device which uses a probe to measure the IOP by tonometry) IOP was then monitored after the first dose (10 μg Exenatide or saline placebo administered via subcutaneous injection) throughout the 24 hour baseline visit. A second dose is administered on day 2 (10 μg Exenatide or saline placebo).

Following the baseline visit the groups proceed for a further 3 months receiving either twice daily Exenatide 10 μg or placebo as per initial randomization. Intraocular pressure (IOP) was measured again at 12 weeks.

The IOP measurement procedures caused a small amount of discomfort but no analgesia was required and the procedure was repeatable over the course of the study visits.

Central corneal thickness (CCT) was measured at baseline in order to interpret the IOP readings. This was measured using a pachymeter following topical anaesthesia of the cornea as is usual practice.

### Results

TABLE 1
Baseline characteristics of participants

| All | | |
|---|---|---|
| | Median (range) | Mean (SD) |

| Number | 15 | | |
|---|---|---|---|
| Age | 25 | (18, 57) | 28 (9) |
| Gender (% female) | 100 | | |
| BMI (kg/m2) | 37.1 | (29.4, 54.4) | 38.1 (6.2) |

TABLE 2
IOP measurements (12 week data)

| | | | Difference | Difference between arms at 12 weeks | |
|---|---|---|---|---|---|
| | | | baseline to | | Hierarchical |
| Intraocular | | | 12 weeks | t-test | regression |
| pressure | Baseline | 12 weeks | mean (SE); | mean (SE); | mean (SE); |
| (mmHg) | mean (SD), n | | 95% CI, p | 95% CI, p | 95% CI, p |
| Exenatide | 18.0 (2.0), | 16.9 (1.7), | −1.2 (0.8); | 0.0 (1.1); | −0.1 (1.1); |
| | n = 7 | n = 7 | (−3.8, 1.4), | (−2.1, 2.1), | (−2.3, 2.1), |
| | | | p = 0.306 | p = 0.994 | p = 0.910 |
| Placebo | 16.7 (2.6), | 16.9 (1.9), | 0.5 (0.6); | | |
| | n = 8 | n = 7 | (−0.7, 1.7), | | |
| | | | p = 0.375 | | |

TABLE 3
IOP measurements (first day data)

| | Difference | Difference | Difference | Difference |
|---|---|---|---|---|
| | baseline | baseline | baseline | baseline |
| | to 2.5 h | to 6 h | to 11 h | to 23 h |
| | mean (SE); | mean (SE); | mean (SE); | mean (SE); |
| | [95% CI] | [95% CI] | [95% CI] | [95% CI] |
| IOP (mmHg) | −0.9 (0.6); | −1.9 (0.9); | −1.9 (1.1); | −1.4 (0.7); |
| Exenatide | [−2.2, 0.3] | [−3.8, −0.1] | [−4.1, 0.3] | [−2.8, 0.0] |
| IOP (mmHg) | 0.2 (0.4); | 0.9 (0.5); | −0.3 (0.8); | 0.9 (0.6); |
| Placebo | [−0.7, 1.0] | [0.0, 1.9] | [−1.8, 1.2] | [−0.3, 2.1] |

Both patient groups recorded normal levels of IOP at the baseline, i.e. prior to administration of exenatide or placebo (see Table 2).

After 12 weeks patients treated with systemic exenatide had reduced IOP by approximately 7% from baseline, whereas patients treated with the placebo had slightly increased IOP (Table 2 and FIG. 1).

Table 3 and FIG. 2 show the difference from baseline in both groups at various time points in the 24 hours following dosing with the first dose at the baseline visit (t=0 h).

These data suggest the use of exenatide to reduce IOP in a disease population with raised IOP would be beneficial. Furthermore, it is envisaged that topical application will have particular benefits.

### REFERENCES

1. Quigley H A. “Number of people with glaucoma worldwide”, British Journal of Ophthalmology, 1996: 90: 389-393.
2. Khaw P T, Shah P, Elkington A R. “Glaucoma—1: Diagnosis”, BMJ, 2004; 328: 97-99.
3. Inoue K. “Managing adverse effects of glaucoma medications”, Clin. Opthalmol. 2014; 8: 903-913.
4. Baggio L L, Drucker D J. “Biology of incretins: GLP-1 and GIP”, Gastroenterology, 2007; 132: 2131-2157.
5. Campbell J E, Drucker D J. “Pharmacology, physiology, and mechanisms of incretin hormone action”, Cell Metab. 2013; 17: 819-837.
6. Larsen P J, Tang-Christensen M, Holst J J, et al. “Distribution of glucagon-like peptide and other preproglucagon-derived peptides in the rat hypothalamus and brainstem”, Neuroscience, 1997; 77: 257-270.
7. Astrup A, Rossner S, Van Gaal L, et al. “NN8022-1807 study group, effects of liraglutide in the treatment of obesity: a randomized, double-blind, placebo-controlled study”, Lancet, 2009; 374:1606-1616.
8. Alhadeff A L, Grill J H. “Hindbrain nucleus tractus solitarius glucagon-like peptide-1 receptor signalling reduces appetitive and motivational aspects of feeding”, Am. J. Physiol. Regul. Integr. Comp. Physiol. 2014; 307: R465-470.
9. Flint A, Raben A, Astrup A, et al. “Glucagon-like peptide-1 promotes satiety and suppresses energy intake in humans”, J. Clin. Invest. 1998; 101: 515-520.
10. Alvarez E, Roncero I. Chowen J A, et al. “Expression of the glucagon-like peptide-1 receptor gene in rat brain”, J. Neurochem. 1996; 66: 920-927.
11. Cork S C, Richards J E, Holt M K, et al. “Distribution and characterisation of glucagon-like peptide-1 receptor expressing cells in the mouse brain”, Mol. Metab. 2015; 4: 718-731.
12. Marques F, Sousa J C, Coppola G, et al. “Transcriptome signature of the adult mouse choroid plexus”, Fluids Barriers CNS, 2011; 8: 10.
13. WO 2016/034851 A1
14. Botfield H F, Uldall M S, Westgate C S J, et al. “A glucagon-like peptide-1 receptor agonist reduces intracranial pressure in a rat model of hydrocephalus”, Sci. Transl. Med. 2017; 9: 1-11.
15. Machiele R, Motlagh M, Patel B C. “Intraocular Pressure”, StatPearls. Treasure Islan (FL): StatPearls Publishing; 2020.
16. “Terminology and guidelines for glaucoma”, European Glaucoma Society, 4^{th} edition, Ed. PubliComm (2014).
17. “Glaucoma: diagnosis and management”, NICE guidelines, November 2017.
18. Liu J H K, Zhang X, Kripke D F, et al. “Twenty-four-hour intraocular pressure pattern associated with early glaucomatous changes”, Investigative Ophthalmology & Visual Science, 2003; 44: 1596-1590.
19. Stewart W C, GeigerAC, Jenkins J N. “The benefit of repeated intraocular pressure measurements in clinical trials”, Arch Ophthalmol. 2004; 122: 936-937.
20. Stewart W C, Stewart J A, Nelson L A, et al. “Mean standard deviations for common glaucoma treatments”, Acta Ophthamologica, 2009; 87: 112-114.
21. Sihota R, Angmo D, Ramaswamy D, et al. Indian J. Ophthamol. 2018; 66: 499-505.
22. Nucci C, Martucci A, Giannini C, et al. “Neuroprotective agents in the management of glaucoma”, Eye, 2018; 32: 938-945.
23. Vasudevan S K, Gupta V, Crowston J G, “Neuroprotection in glaucoma”, Indian J. Ophthamol. 2011; 59: S102-S113.

1. A method for reducing intraocular pressure (IOP) in a subject suffering a disorder associated with intraocular pressure, the method comprising administering a GLP-1 receptor agonist, or a pharmaceutically acceptable salt thereof, to the subject.

2. The method according to claim 1, wherein the intraocular pressure to be reduced is elevated intraocular pressure.

3. The method according to claim 1, wherein the disorder associated with intraocular pressure is selected from Open-angle glaucoma, Ocular hypertension, Chronic open angle glaucoma, Primary angle closure glaucoma, Acute angle closure glaucoma, Angle closure glaucoma, Normal tension glaucoma (or Low tension glaucoma), Exfoliation syndrome, Pseudoexfoliation syndrome, Exfoliation glaucoma, Pigment dispersion syndrome, Primary congenital glaucoma, Nail-Patella syndrome, Secondary congenital glaucoma, Childhood glaucoma secondary to intraocular surgery, Glaucoma secondary to retinopathy of prematurity, Pupil block glaucoma, Glaucoma associated with anterior segment dysgenesis, Glaucoma associated with disorders of the corneal endothelium, Iridocorneal endothelial syndrome (ICE syndrome), Chandler syndrome, Essential/Progressive Iris Atrophy, Iris Nevus/Cogan-Reese Syndrome, Glaucoma associated with disorders of the iris, Plateau iris, Glaucoma associated with disorders of the ciliary body, Glaucoma associated with disorders of the lens, Glaucoma associated with disorders of the retina, Glaucoma associated with disorders of the vitreous, Glaucoma associated with disorders of the choroid, Glaucoma associated with elevated episcleral venous pressure, Glaucoma associated with intraocular tumours, Glaucoma associated with ocular inflammation, Glaucoma associated with systemic inflammation, Glaucoma associated with systemic disease, Steroid-induced glaucoma, Glaucoma associated with medications, Glaucoma associated with intraocular haemorrhage, Neovascular glaucoma, Glaucoma associated with ocular trauma, Glaucoma secondary to angle recession, Glaucoma secondary to cyclodialysis cleft, Glaucoma after ocular surgery and Aqueous misdirection.

4. The method according to claim 1, wherein the disorder associated with intraocular pressure is selected from ocular hypertension, open-angle glaucoma, primary angle closure glaucoma, angle closure glaucoma, and normal tension glaucoma.

5. The method according to claim 1, wherein the GLP-1 receptor agonist is selected from exendins, exendin analogs, GLP-1(7-36)amide, GLP-1(7-36)amide analogs, GLP-1(7-37), and GLP-1(7-37)analogs.

6. The method according to claim 5, wherein the GLP-1 receptor agonist is selected from exendin-4, exenatide, liraglutide, lixisenatide, albiglutide, dulaglutide, and semaglutide.

7. The method according to claim 5, wherein the GLP-1 receptor agonist is selected from exendin-4 and exenatide.

8. The method according to claim 1, wherein the method further comprises determining baseline IOP of the subject prior to administration of the GLP-1 receptor agonist, and wherein the GLP-1 receptor agonist reduces IOP in the subject by at least 5% from baseline.

9. The method according to claim 1, wherein the method further comprises determining the baseline IOP of the subject prior to administration of the GLP-1 receptor agonist, and wherein the GLP-1 receptor agonist reduces IOP in the subject by at least 5% from baseline for at least 3 hours after administration.

10. The method according to claim 1, wherein the method further comprises determining baseline IOP of the subject prior to administration of the GLP-1 receptor agonist, and wherein the GLP-1 receptor agonist reduces IOP in the subject by at least 5% from baseline after 60 minutes from dosing.

11. The method according to claim 1, wherein the method further comprises determining the baseline IOP of the subject prior to administration of the GLP-1 receptor agonist, and wherein the GLP-1 receptor agonist reduces IOP in the subject by at least 1 mmHg from baseline.

12. The method according to claim 1, wherein the GLP-1 receptor agonist is administered by oral, sublingual, buccal, nasal, intra-arterial, intra-articular, intracardiac, intradermal, intramuscular, intraocular, intrathecal, intravenous, intravitreal, intraventricular, subcutaneous, subconjunctival, retrobulbar, peribulbar, intracameral, or topical administration.

13. The method according to claim 1, wherein the GLP-1 receptor agonist is administered topically to an eye of the subject.

14. The method according to claim 1, wherein the GLP-1 receptor agonist is administered by subcutaneous or intravenous administration.

15. (canceled)

16. The method according to claim 1, wherein the GLP-1 receptor agonist is administered at a dose of 0.01 to 100 μg.

17. The method according to claim 1, wherein the GLP-1 receptor agonist is administered once or twice a daily.

18. (canceled)

19. (canceled)

20. The method according to claim 1, wherein the GLP-1 receptor agonist is administered in combination with one or more therapeutic agents selected from adrenergic agonists, adrenergic antagonists, carbonic anhydrase inhibitors, cholinergic agents, prostaglandin derivates, phenoxyacetic acid derivatives, steroid antagonists, atrial natriuretic peptides, angiotensin converting enzyme inhibitors, ocular hypotensive lipids, neuroprotective agents, Rho kinase inhibitors, nitric oxides, and beta blockers.

21. The method according to claim 1, wherein the GLP-1 receptor agonist further provides a neuroprotective effect.

22. The method according to claim 1, wherein the subject is a human.

23. (canceled)

24. The method according to claim 1, wherein the GLP-1 receptor agonist, or a pharmaceutically acceptable salt thereof, is administered to the subject in a pharmaceutical composition comprising the GLP-1 receptor agonist and at least one pharmaceutically acceptable excipient.

25-28. (canceled)
